(19) United States
(12) Patent Application Publication
(10) Pub. No.: US 2014/0277279 A1
(43) Pub. Date: Sep. 18, 2014

(54) Methods And Associated Algorithms For Programming A Baroreflex Activation Therapy Device

(71) Applicant: CVRX, INC., Minneapolis, MN (US)

(72) Inventors: Adam Cates, Minneapolis, MN (US); Eric Grant Lovett, Mendota Heights, MN (US); Seth Wilks, Maple Grove, MN (US)

(21) Appl. No.: 13/842,699

(22) Filed: Mar. 15, 2013

(51) Int. Cl. A61N 1/36; A61N 1/372

(52) U.S. Cl. CPC A61N 1/36189; A61N 1/3606; A61N 1/37235
USPC 607/59

(57) ABSTRACT

Methods and associated devices and algorithms for programming a therapy for an implanted baroreflex activation system are described and may include determining one or more programmable operating parameters of the therapy which do not cause a patient to experience extraneous stimulation associated with the therapy and programming the implanted baroreflex stimulation system with programmable operating parameters.

### FIELD OF THE INVENTION

The present invention relates generally to implantable baroreflex activation therapy devices, and more particularly the present invention relates to devices and methods for programming and/or adjusting operating parameters of such therapy devices.

### BACKGROUND OF THE INVENTION

Cardiovascular disease is a major contributor to patient illness and mortality. It also is a primary driver of health care expenditure, costing billions of dollars each year in the United States. Hypertension, or high blood pressure, is a major cardiovascular disorder that is estimated to affect 65 million people in the United Sates alone. Hypertension is a leading cause of heart failure and stroke, is the primary cause of death for tens of thousands of patients per year, and is listed as a primary or contributing cause of death for hundreds of thousands of patients per year in the U.S. Accordingly, hypertension is a serious health problem demanding significant research and development for the treatment thereof.

One method of treating hypertension comprises stimulation of baroreceptors, a practice known as baroreflex activation therapy. Baroreceptors are sensory nerve ends that are profusely distributed within the arterial walls of the major arteries, as well in the heart, aortic arch, carotid sinus or arteries, and in the low-pressure side of the vasculature such as the pulmonary artery and vena cava. Baroreceptor signals are used to activate a number of body systems which collectively may be referred to as the baroreflex system. Baroreceptors are connected to the brain via the nervous system. Thus, the brain is able to detect changes in blood pressure, which can be related to, or indicative of, cardiac output.

Heart failure is the final common expression of a variety of cardiovascular disorders, including ischemic heart disease. In many cases it can be characterized by an inability of the heart to pump enough blood to meet the body's needs (insufficient cardiac output) and results in fatigue, reduced exercise capacity and poor survival. Heart failure results in the activation of a number of body systems to compensate for the heart's inability to pump sufficient blood. Many of these responses are mediated by an increase in the level of activation of the sympathetic nervous system, as well as by activation of multiple other neurohormonal responses. Generally speaking, this sympathetic nervous system activation (also known as a pressor response) signals the heart to increase heart rate and force of contraction to increase the cardiac output; it signals the kidneys to expand the blood volume by retaining sodium and water; and it signals the arterioles to constrict to elevate the blood pressure. The cardiac, renal and vascular responses increase the workload of the heart, further accelerating myocardial damage and exacerbating the heart failure state.

Baroreflex activation therapy (“BAT”) works by artificially activating the carotid sinus baroreflex. U.S. Pat. No. 6,522,926 to Kieval, et al. discloses a baroreflex activation system and method for activating baroreceptors to regulate blood pressure for the treatment of hypertension and/or heart failure (to counteract the above-described pressor response). Generally speaking, the baroreceptor activation device may be activated, deactivated or otherwise modulated to activate one or more baroreceptors and induce a baroreceptor signal or a change in the baroreceptor signal to thereby affect a change in the baroreflex system. The baroreceptor activation device may be activated, deactivated, or otherwise modulated continuously, periodically, or episodically. The baroreceptor activation device may utilize electrical as well as mechanical, thermal, chemical, biological, or a combination thereof to activate the baroreceptor. The baroreceptor may be activated directly, or activated indirectly via the adjacent vascular tissue.

Activating the baroreflex (by, for example, stimulation of baroreceptors) increases afferent electrical signals from the source (baroreceptor) through the carotid sinus nerve (Hering's nerve, a branch of the glossopharyngeal nerve, cranial nerve IX) to the medullary brain centers that regulate autonomic tone. Increased afferent signals to these medullary centers cause a reduction in sympathetic tone and an increase in parasympathetic tone. This results in lower heart rate, reduced sodium and water reabsorption by the kidney resulting in a diuresis, relaxation of the smooth muscle in the blood vessels which results in vasodilatation and a reduction in blood pressure. Thus, peripheral activation of the baroreflex results in a physiologic response whereby blood pressure is controlled by mechanisms determined by the integrative action of the central nervous system action on all peripheral organs and blood vessels.

The process of implanting a baroreflex activation device for delivering baroreflex therapy, such as an electrode assembly, involves positioning the assembly such that one or more electrodes are properly situated against, about or otherwise in contact with the wall of the vessel containing baroreceptors (for example, the carotid sinus), and securing the electrode assembly so that the positioning is maintained. The process of adjusting and re-adjusting the position of the electrode assembly during implantation, known as mapping, adds to the overall procedure time. Present-day procedures involve positioning and holding the electrode assembly in place with forceps, hemostat or other suitable tool while applying the stimulus and observing the response in the patient. Movement by as little as 1 mm can make a medically relevant difference in the effectiveness of the baroreceptor activation.

One example of mapping methods and techniques for implanting electrodes is disclosed in U.S. Pat. No. 6,850,801 to Kieval et al. The positioning is a critical step, as the electrodes must direct as much energy as possible toward the baroreceptors for maximum effectiveness and efficiency. The energy source for the implanted baroreflex stimulation device is typically an on-board battery with finite capacity, and it is desirable to provide a lower energy source to ensure patient safety. A high-efficiency implantation will provide a longer battery life and correspondingly longer effective service life between surgeries because less energy will be required to achieve the needed degree of therapy. As such, during implantation of the electrode assembly, the position of the assembly is typically adjusted several times during the implantation procedure in order to optimize the baroreflex response. Further procedures and related devices are described in U.S. Published Patent Application No. 2012/0109250 to Cates et al.

After the device has been successfully implanted, a number of operating parameters may be adjusted and/or tested in order to tailor operation of the device to the particular patient. Ideally, the operating parameters of the device are adjusted to provide a desired therapeutic effect, while minimizing any side effects attributable to operation of the device. Further, because the device includes a dedicated power supply having a finite energy amount, it is desirable to adjust the operating parameters to prolong the service life of the device to avoid the cost and trauma associated with replacing a battery of a device (or entire device) due to a depleted power supply. Programming the device, therefore, involves a tradeoff between obtaining a desired response, minimizing or preventing side effects, and prolonging the service life of the power supply of the device. The number of adjustable operating parameters, and the range of possible settings for each parameter represents a staggeringly large number of possible combinations which can be tested. One or more hemodynamic responses of the patient are measured for each combination tested, and a resting period is typically required between tests of various parameter combinations. Accordingly, programming the device can be a very lengthy and burdensome procedure.

An opportunity therefore exists to improve upon current techniques to provide simpler, faster, safer and/or more effective methods of programming an implanted baroreflex activation device for delivery of chronic therapy.

### SUMMARY OF THE INVENTION

In one embodiment, the present invention comprises a method of programming a therapy for an implanted baroreflex stimulation system, the implanted baroreflex stimulation system configured to deliver the therapy to a patient, the therapy having at least two programmable operating parameters. The method comprises determining a first programmable operating parameter of the therapy, including delivering a first therapy pulse to the patient, the therapy pulse including initial settings for each of the at least two programmable operating parameters, increasing the first programmable operating parameter in increments, delivering subsequent therapy pulses at each increment, determining, for at least some of the therapy pulses, whether the patient is experiencing extraneous stimulation associated with the therapy pulses, and selecting a value for the first programmable operating parameter based at least in part on the last therapy pulse delivered which did not cause the patient to experience extraneous stimulation associated with the therapy pulse.

The method further comprises determining a second programmable operating parameter of the therapy, including delivering a further therapy pulse to the patient, the therapy pulse including the selected value for the first programmable operating parameter and the initial setting for the second programmable operating parameter, increasing the second programmable operating parameter in increments, delivering subsequent therapy pulses at each increment, determining, for at least some of the therapy pulses, whether the patient is experiencing extraneous stimulation associated with the therapy pulses, and selecting a value for the second programmable operating parameter based at least in part on the last therapy pulse delivered which did not cause the patient to experience extraneous stimulation associated with the therapy pulse. The method further comprises programming the implanted baroreflex stimulation system with the selected values for the first and second programmable operating parameters.

In another embodiment, the present invention comprises a method of programming a therapy for an implanted baroreflex stimulation system, the system configured to deliver the therapy to a patient and including a subcutaneously-implanted pulse generator and a baroreflex activation device in the form of an electrode structure coupled to the pulse generator via a lead, the baroreflex activation device implanted such that the electrode structure is proximate a baroreceptor of a patient, the therapy having at least two programmable operating parameters, the method comprising causing a programming device to be manufactured and made available to a user, the programming device communicable with the implanted baroreflex stimulation system, and providing instructions to the user for programming.

In another embodiment, the present invention comprises a programming device for programming a therapy for an implanted baroreflex stimulation system, the implanted baroreflex stimulation system configured to deliver the therapy to a patient, the therapy having at least two programmable operating parameters, the programming device communicably coupled to the implanted baroreflex stimulation system and configured to determine a first programmable operating parameter of the therapy, determine a second programmable operating parameter of the therapy, and program the implanted baroreflex stimulation system with the selected values for the first and second programmable operating parameters.

### BRIEF DESCRIPTION OF THE DRAWINGS

The invention may be more completely understood in consideration of the following detailed description of various embodiments of the invention in connection with the accompanying drawings, in which:

FIG. 1 is a cross-sectional schematic illustration of baroreceptors within the vascular wall and the baroreflex system.

FIG. 2 is a schematic illustration of a baroreceptor activation system in accordance with the present invention.

FIGS. 3A and 3B are schematic representations of a baroreflex activation system according to an embodiment of the present invention implanted on a carotid sinus within a patient.

FIGS. 4A and 4B are schematic representations of a baroreflex activation system in the form of an external conductive structure configured to electrically induce a baroreceptor signal in accordance with an embodiment of the present invention.

FIGS. 5A and 5B are schematic representations of a baroreflex activation system in the form of an internal conductive structure configured to electrically induce a baroreceptor signal in accordance with an embodiment of the present invention.

FIG. 6 is a schematic flowchart of a programming procedure according to an embodiment of the present invention.

FIG. 7 is a detailed schematic flowchart of one portion of the programming procedure of FIG. 6.

FIG. 8 is a detailed schematic flowchart of another portion of the programming procedure of FIG. 6.

FIG. 9 is a detailed schematic flowchart of another portion of the programming procedure of FIG. 6.

FIG. 10 is a detailed schematic flowchart of another portion of the programming procedure of FIG. 6.

FIG. 11 is a schematic graphic user interface for according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

To address the problems of hypertension, heart failure, other cardiovascular disorders and renal disorders, the present invention basically provides a number of devices, systems and methods by which the baroreflex system is activated to reduce excessive blood pressure, autonomic nervous system activity and neurohormonal activation. In particular, the present invention provides a number of devices, systems and methods by which baroreceptors may be activated, thereby indicating an increase in blood pressure and signaling the brain to reduce the body's blood pressure and level of sympathetic nervous system and neurohormonal activation, and increase parasympathetic nervous system activation, thus having a beneficial effect on the cardiovascular system and other body systems.

For general information pertaining to the cardiovascular, circulatory and nervous systems, as well as baroreceptor and baroreflex therapy systems that may be used in whole or in part with embodiments of the present invention, reference is made to the following commonly assigned patent applications and patents: US Published Patent Application Nos. 2006/0004417 to Rossing et al., 2006/0074453 to Kieval et al., 2008/0082137 to Kieval et al. and 2012/0109250 to Cates et al., and U.S. Pat. Nos. 6,522,926, 6,850,801, 6,985,774, 7,480,532 and 7,499,747 all to Kieval et al., U.S. Pat. No. 7,499,742 to Bolea et al., U.S. Pat. No. 7,835,797 to Rossing et al., U.S. Pat. No. 7,848,812 to Crowley et al., U.S. Pat. No. 8,086,314 to Kieval, and U.S. Pat. No. 8,150,521 to Crowley et al., the disclosures of which are hereby incorporated by reference in their entireties except for the claims and any express definitions.

Refer now to FIG. 1, which depicts a schematic illustration of baroreceptors 30 disposed in a generic vascular wall 40 and a schematic flow chart of the baroreflex system 50. Baroreceptors 30 are profusely distributed within the vascular walls discussed previously, and generally form an arbor 32. The baroreceptor arbor 32 comprises a plurality of baroreceptors 30, each of which transmits baroreceptor signals to the brain 52 via nerve 38. The baroreceptors 30 are so profusely distributed and arborized within the vascular wall 40 that discrete baroreceptor arbors 32 are not readily discernable. To this end, those skilled in the art will appreciate that the baroreceptors 30 depicted in FIG. 1 are primarily schematic for purposes of illustration and discussion.

Baroreceptor signals in the arterial vasculature are used to activate a number of body systems which collectively may be referred to as the baroreflex system. For the purposes of the present invention, it will be assumed that the “receptors” in the venous and cardiopulmonary vasculature and heart chambers function analogously to the baroreceptors in the arterial vasculature, but such assumption is not intended to limit the present invention in any way. In particular, the methods described herein will function and achieve at least some of the stated therapeutic objectives regardless of the precise and actual mechanism responsible for the result. Moreover, the present invention may activate baroreceptors, mechanoreceptors, pressoreceptors, stretch receptors, chemoreceptors, or any other venous, heart, or cardiopulmonary receptors which affect the blood pressure, nervous system activity, and neurohormonal activity in a manner analogous to baroreceptors in the arterial vasculation. For convenience, all such venous receptors will be referred to collectively herein as “baroreceptors” or “receptors” unless otherwise expressly noted.

While there may be small structural or anatomical differences among various receptors in the vasculature, for the purposes of some embodiments of the present invention, activation may be directed at any of these receptors and/or nerves and/or nerve endings from these receptors so long as they provide the desired effects. In particular, such receptors will provide afferent signals, i.e., signals to the brain, which provide the blood pressure and/or volume information to the brain. This allows the brain to cause “reflex” changes in the autonomic nervous system, which in turn modulate organ activity to maintain desired hemodynamics and organ perfusion. Stimulation of the baroreflex system may be accomplished by stimulating such receptors, nerves, nerve fibers, or nerve endings, or any combination thereof.

Generally, implantable baroreflex activation systems include a control system, a baroreceptor activation device, one or more optional sensors, and an optional programming device. The baroreceptor activation device may comprise a wide variety of devices which utilize mechanical, electrical, thermal, chemical, biological, or other means to activate one or more baroreceptors. The baroreceptor may be activated directly, or activated indirectly via the adjacent vascular tissue. According to various embodiments, the baroreceptor activation device may be positioned intravascularly (e.g., inside the vascular lumen); extravascularly (outside of the vessel, e.g., disposed on the outer surface of the vessel, or in contact with at least a portion of the vessel, or proximate the vessel); transvascularly (e.g., at least a portion of the device being intravascular and at least a portion of the device being extravascular, or at least a portion of the device being in a first vessel and at least a portion of the device being in a second, neighboring vessel); intramurally (e.g., within the vascular wall); or around all or a portion of a vascular sheath structure surrounding at least one vein and one artery as well as associated nerve structures; or otherwise positioned proximate tissue in which baroreceptors reside.

Referring now to FIG. 2, a baroreflex activation system according to an embodiment of the present invention is depicted which includes a control system 60, one or more baroreceptor activation devices 70 in the form of an electrode structure connected to the control system via an electrical lead 72, and an input device (programmer) 64 configured to communicate with the control system. The control system includes a therapy block 61 comprising one or more associated processors 63, and a memory 62 adapted to store one or more algorithms which define a stimulus (or therapy) regimen which dictates the characteristics of the control signal as a function of time, and thus dictates the stimulation of baroreceptors as a function of time. Control system 60 includes a driver 66 to provide the desired power mode for the baroreceptor activation device 70. For example if the baroreceptor activation device 70 utilizes electrical actuation, the driver 66 may comprise a power amplifier, pulse generator or the like to selectively deliver electrical control signals. As depicted in FIG. 2, control system 60, activation device 70, lead 72 and optional sensor 80 are configured to be implanted within the patient, while programmer device 64 and display 65 are configured to be external to the patient and operably communicate with control system 60 through wireless or wired connections. In another embodiment, programmer device 64 includes a display 65.

In one embodiment in which driver 66 comprises a pulse generator, control system 60 may simply be referred to as the pulse generator, which is understood to encompass not only driver 66 but also the necessary circuitry and components for generating and delivering control signals to the baroreceptor activation device. The control signal may alternately be referred to as an output signal, a therapy signal, a therapy output signal, or simply a pulse. Such a pulse generator and associated circuitry may be enclosed within a hermetically sealed housing configured for implantation into the patient. The pulse generator housing may include a header adapted to facilitate connection to one or more leads, as well as an indifferent case electrode 67 on an outer surface of the housing.

The control signal generated by the control system may be continuous, periodic, episodic or a combination thereof, as dictated by the one or more algorithms. Continuous control signals include a pulse, a train of pulses, a triggered pulse and a triggered train of pulses, all of which are generated continuously. Examples of periodic control signals include each of the continuous control signals described above which have a designated start time (e.g., beginning of each minute, hour or day) and a designated duration (e.g., 1 second, 1 minute, 1 hour). Examples of episodic control signals include each of the continuous control signals described above which are triggered by an episode (e.g., activation by the patient/physician, an increase in blood pressure above a certain threshold as measured by a sensor, etc.). The therapy (or stimulus) regimen may refer to characteristics of the control signal as a function of time, on a larger time scale than that of the control signal itself. For example, a therapy regimen may dictate the control signal based on a time of day, based on an episode or event, or based on feedback.

In one embodiment, the pulse may be monophasic. In one embodiment, the pulse is multiphasic wherein the amplitude, duration, polarity and/or waveform shape of each phase may be identical to, or different from, one or more of the other phases. Multiphasic pulses may also include time delays between pulses, referred to as an interphase delay. Such interphase delays may be identical between phases, or different between phases.

The power level of the baroreceptor activation device may be altered by changing the voltage, current and/or one or more characteristics of the output signal. The control signal of the baroreceptor activation device may be, for example, constant current or constant voltage. For example, in an embodiment wherein the control signal comprises a series of pulses (a pulse train), several pulse characteristics may be changed individually or in combination to change the power or energy level of the output signal. Such pulse characteristics include, but are not limited to: pulse amplitude (PA), pulse frequency (PF), pulse width or duration (PW), pulse waveform (square, triangular, sinusoidal, etc.), pulse polarity and pulse waveform (monophasic, biphasic).

In one embodiment wherein the control signal comprises a pulse train, several other signal characteristics may be changed in addition to the pulse characteristics described above. The control signal may comprise a pulse train which generally includes a series of pulses occurring in bursts. Pulse train characteristics which may be changed include, but are not limited to: pulse frequency (PF) or interval between pulses, burst duration (BD) and burst interval (BI). In an embodiment, the burst duration equals the burst interval and the series of pulses occurs continuously. The signal or a portion thereof (e.g., burst within the pulse train) may be triggered by any of the events discussed previously, or by a particular portion of the arterial pressure signal or an ECG signal or another physiologic timing indicator. If the signal or a portion thereof is triggered, the triggering event may be changed and/or the delay from the triggering event may be changed.

Output signal characteristics for a baroreflex therapy system having a monopolar electrode according to an embodiment of the present invention differ from prior approaches, and provide greater stimulation efficacy with lower power consumption. For example, suitable pulse widths for the present invention are in the range of about 5 microseconds (μs) to 5000 μs, preferably in the range of about 30-500 μs, and more preferably in the range of about 60-150 μs. Suitable pulse amplitudes are in the range of about 1-50 milliamps (mA), preferably in the range of about 2-20 mA, and more preferably in the range of about 3-10 mA. Suitable pulse frequencies are in the range of about 2-200 Hz, preferably in the range of about 10-100 Hz, and more preferably in the range of 40-80 Hz.

Referring now to FIGS. 3A-5B, different embodiments of electrode structures are depicted. In one embodiment depicted in FIGS. 3A and 3B, baroreceptor activation device 70 comprises an electrode structure 110. Electrode structure 110 generally includes an electrode mounted on, integrated with, or otherwise coupled to a backer structure. The electrode may comprise platinum iridium, and may include a surface treatment, such as iridium oxide or titanium nitride and/or can include steroid, anti-inflammatory, antibiotic and/or analgesic compounds, for example. Further details about such an electrode structure may be found in US Published Application No. 2012/0109250 to Cates et al., incorporated by reference above. As used herein, the term “baroreceptor activation device” may refer to the electrode, or the electrode structure, or the electrode structure in combination with the lead, or the entire system.

In another embodiment depicted in FIGS. 4A-4B, baroreceptor activation device 300 is depicted in the form of an extravascular electrically conductive structure or electrode 302. The electrode structure 302 may comprise a coil, braid or other structure capable of surrounding the vascular wall. Alternatively, the electrode structure 302 may comprise one or more electrode patches distributed around the outside surface of the vascular wall. The extravascular electrode structure 302 may receive electrical signals directly from the driver 66 of the control system 60 by way of electrical lead 304.

Refer now to FIGS. 5A and 5B which show schematic illustrations of a baroreceptor activation device 280 in the form of an intravascular electrically conductive structure or electrode 282. The electrode structure 282 may comprise a self-expanding or balloon expandable coil, braid or other stent-like structure disposed in the vascular lumen. The electrode structure 282 may serve the dual purpose of maintaining lumen patency while also delivering electrical stimuli. To this end, the electrode structure 282 may be implanted utilizing conventional intravascular stent and filter delivery techniques. Preferably, the electrode structure 282 comprises a geometry which allows blood perfusion therethrough. The electrode structure 282 comprises electrically conductive material which may be selectively insulated to establish contact with the inside surface of the vascular wall 40 at desired locations, and limit extraneous electrical contact with blood flowing through the vessel and other tissues. The electrode structure 282 is connected to electric lead 284 which is connected to pulse generator 66 of the control system 60. As mentioned previously, the electrical control signal generated by the driver 66 may be continuous, periodic, episodic or a combination thereof, as dictated by an algorithm contained in memory 62 of the control system 60.

Various embodiments of the inventive devices may be entirely intravascular, entirely extravascular, or partially intravascular and partially extravascular. Furthermore, devices may reside wholly in or on arterial vasculature, wholly in or on venous vasculature, or in or on some combination of both. In some embodiments, for example, implantable devices may positioned within an artery or vein, while in other embodiments devices may be placed extravascularly, on the outside of an artery or vein. In introducing and placing devices of the present invention, any suitable technique and access route may be employed. For example, in some embodiments an open surgical procedure may be used to place an implantable device. Alternatively, an implantable device may be placed within an artery or vein via a transvascular, intravenous approach. In still other embodiments, an implantable device may be introduced into vasculature via minimally invasive means, advanced to a treatment position through the vasculature, and then advanced outside the vasculature for placement on the outside of an artery or vein. For example, an implantable may be introduced into and advanced through the venous vasculature, made to exit the wall of a vein, and placed at an extravascular site on an artery.

After a baroreflex activation system has been successfully implanted, a physician or other medical professional may utilize a programming device to enable operation of the system and adjust one or more operating parameters of the baroreflex activation system to suit the particular patient. Operating parameters may also be referred to as regimen parameters, therapy parameters, therapy characteristics, signal characteristics, pulse characteristics or other similar terms as will be apparent to one skilled in the art for describing parameters or characteristics which impact or effect therapy efficacy and/or device longevity. It has been found by the present inventors that by focusing on adjusting pulse amplitude, pulse width and pulse frequency, a suitable stimulus regimen can be created for the particular patient and saved within the implanted control system. Absent any limitations, a stimulus regimen could be created based on an infinite number of combinations of pulse amplitude, pulse width, and pulse frequency. However, known limitations include limitations of the device, extraneous stimulation limits, and practical consideration of battery life.

For a given baroreflex activation system, the pulse amplitude at a given pulse width is constrained by impedances of the electrode, the lead, internal circuitry of the implantable pulse generator, or other factors, thereby creating an upper limit to suitable pulse amplitude values. Similarly, the anatomy of each individual patient and the placement of the implanted electrode will limit the available combinations of pulse amplitude and pulse width which can be used. Exceeding these limits can create extraneous stimulation (e.g., stimulation of other nerves, nerve fibers, or other excitable tissues) which not only reduces efficacy of the baroreflex therapy but typically creates pain or discomfort to the patient. Finally, because the implanted baroreflex activation system is powered by a dedicated battery having a finite energy amount, it is desirable to prolong the service life of the system as much as practical, to avoid or delay the cost and trauma associated with replacing a system having a depleted power supply. While a stimulus regimen programmed with relatively large pulse amplitudes generally results in a greater therapeutic effect (i.e., efficacy), such large pulse amplitude signals generally result in decreased battery life.

Therefore, a suitable stimulus regimen is one which is within the compliance limits of the characteristics of the baroreflex activation system, which avoids patient discomfort resulting from extraneous tissue stimulation, and which maintains acceptable battery life.

In one embodiment depicted in FIG. 6, a method 200 of programming a baroreflex activation device is depicted which begins in step 202 by obtaining baseline patient hemodynamic measurements such as blood pressure (systolic, diastolic, peripheral or central), heart rate, respiration, posture, electrocardiogram, blood volumetric flow rate, blood flow velocity, blood pH, O2 or CO2 content, mixed venous oxygen saturation (SVO2), vasoactivity, nerve activity, tissue activity, or other suitable parameters. Such baseline patient hemodynamic measurements may be obtained with programming device 64, or may be obtained by other means and the measurements manually entered into programming device 64. In step 204, initial operating parameters are programmed into the baroreflex activation device. In step 206, the pulse amplitude is adjusted and set. In step 208, the pulse width is adjusted and set. In step 210, the pulse amplitude and pulse width settings are verified. In step 212, patient hemodynamic measurements are obtained. And in step 214, the pulse frequency is adjusted and set as needed. These steps will now be explained in greater detail.

Referring now to step 204, initial operating parameters are programmed into the control system of the baroreflex activation device. The initial operating parameters may be manually entered into programming device 64 and transmitted to the control system, or the initial operating parameters may be stored in a memory of programming device 64 and accessed by a user to be transmitted to the control system. By way of example, such parameters may comprise nominal values of a pulse amplitude of 1 mA; a pulse width of 30 μs; and a pulse frequency of 80 Hz (pulses per second). In one embodiment, programming device 64 may include historical data relating to past programming procedures for the same patient and/or other patients, wherein the historical data may be relied upon to influence a present programming procedure. Alternately, the implantable baroreflex activation device is provided with initial operating parameters pre-programmed in its memory 62.

Referring now to FIG. 7, step 206 is depicted in further detail. Step 206, adjusting and setting the pulse amplitude, comprises a sequence of sub-steps beginning with step 220, wherein the programming device 64 prompts the control system of the implanted baroreflex activation system to begin delivering therapy via the electrode, which in turn activates the baroreflex system of the patient. This control signal includes the initial operating parameters as described immediately above. In step 222, the patient indicates whether they are experiencing symptoms associated with extraneous stimulation. Such symptoms may include, but are not limited to, pain, twitching, coughing, tickling sensation, muscle contractions, and the like. Alternately, the user of the programming device may observe symptoms indicative of extraneous stimulation without waiting for patient indication of such symptoms. If the patient is not experiencing symptoms of extraneous stimulation, the operating parameters of the baroreflex activation system are verified as being within compliance limits of the system in step 224. Alternatively, step 224 may be omitted, and device compliance verified as part of step 210. If step 224 is skipped, or if the operating parameters of the baroreflex activation system are confirmed to be within compliance limits of the system according to step 224, the programming device 64 is utilized to increase the magnitude of the pulse amplitude in step 226. By way of example only, the magnitude is increased by an increment of 1 mA.

In step 228, programming device 64 prompts the control system of the implanted baroreflex activation system to deliver a control signal to the electrode, which in turn activates the baroreflex system of the patient, wherein the control signal includes the increased pulse amplitude as adjusted in step 226. Subsequent to step 228, steps 222, 224, and 226 are repeated until the magnitude of the pulse amplitude causes extraneous stimulation or exceeds the compliance limits of the baroreflex activation system, at which time programming device 64 is utilized to reduce the magnitude of the pulse amplitude in step 230. By way of example only, the magnitude is decreased in step 230 by an increment of between 0.1 mA and 1 mA, or about 0.5 mA, or about 0.2 mA, or other amounts as determined suitable. In step 232, programming device 64 prompts the control system of the implanted baroreflex activation system to deliver a control signal to the electrode, which in turn activates the baroreflex system of the patient, wherein the control signal includes the decreased pulse amplitude as adjusted in step 230. In step 234, the patient indicates whether they are experiencing symptoms of extraneous stimulation, and optionally the operating parameters of the baroreflex activation system are verified as being within compliance limits of the implanted system. Alternately, the user of the programming device may observe symptoms indicative of extraneous stimulation without waiting for patient indication of such symptoms. If the results of step 234 are not satisfactory, steps 230, 232 and 234 are repeated until the pulse amplitude has been reduced to a magnitude which creates a satisfactory result to step 234; i.e., a pulse amplitude magnitude which does not create extraneous stimulation but is within compliance limits of the implanted system. The programming procedure may then proceed to step 208.

Referring now to FIG. 8, step 208 is described in further detail. In step 250, programming device 64 prompts the control system of the implanted baroreflex activation system to deliver a control signal to the electrode, which in turn activates the baroreflex system of the patient. This control signal includes the initial operating parameters as described immediately above, and the pulse amplitude as determined in step 206 and its associated sub-steps. In step 252, the patient is observed and/or queried whether they are experiencing symptoms of extraneous stimulation. If the patient is not experiencing symptoms of extraneous stimulation, the operating parameters of the baroreflex activation system are verified as being within compliance limits of the system in step 254. Alternatively, step 254 may be omitted, and device compliance verified as part of step 210. If step 254 is skipped, or if the operating parameters of the baroreflex activation system are confirmed to be within compliance limits of the system according to step 254, the programming device 64 is utilized to increase the magnitude of the pulse width in step 256. By way of example only, the magnitude is increased by an increment of 15 μs. Alternately, the magnitude may be adjusted in increments of about 5 μs, or about 30 μs, or about 60 μs, or about 125 μs.

In step 258, programming device 64 prompts the control system of the implanted baroreflex activation system to deliver a control signal to the electrode, which in turn activates the baroreflex system of the patient, wherein the control signal includes the increased pulse width as adjusted in step 256. Subsequent to step 258, steps 252, 254, and 256 are repeated until the magnitude of the pulse width causes extraneous stimulation, exceeds the compliance limits of the baroreflex activation system or causes one of the other operating parameters to exceed the compliance limit, at which time programming device 64 is utilized to reduce the magnitude of the pulse width in step 260 and/or other parameters if needed. By way of example only, the pulse width is decreased in step 260 by an amount between 5 μs and 20 μs, or about 10 μs, or about 15 μs, or other amounts as determined suitable. In step 262, programming device 64 prompts the control system of the implanted baroreflex activation system to deliver a control signal to the electrode, which in turn activates the baroreflex system of the patient, wherein the control signal includes the decreased pulse amplitude as adjusted in step 260. In step 264, the patient is observed and/or queried whether they are experiencing symptoms of extraneous stimulation, and optionally, the operating parameters of the baroreflex activation system are verified as being within compliance limits of the implanted system. If the results of step 264 are not satisfactory, steps 260, 262 and 264 are repeated until the pulse amplitude has been reduced to a magnitude which creates a satisfactory result to step 264; i.e., a pulse width magnitude which does not create extraneous stimulation but is within compliance limits of the implanted system. The programming procedure may then proceed to step 210.

Referring now to FIG. 9, step 210 is depicted in further detail. The operating parameters of the baroreflex activation system as determined in steps 202, 204 and 206 are verified as being within compliance limits of the system in step 270. Optionally, the operating parameters as determined in steps 202, 204 and 206 may also be verified against other criteria as part of step 270, such as a desired charge density limit, or desired device longevity, or desired safety factor/buffer to avoid extraneous stimulation. If the operating parameters are not within compliance limits, the pulse amplitude and/or pulse width may be reduced in steps 272, 274, respectively. For example, in step 272, pulse amplitude may be reduced by an amount between 0.1 mA and 1 mA, or about 0.5 mA, or about 0.2 mA, or other amounts as determined suitable. For example, in step 274, pulse width may be reduced by an amount between 5 μs and 20 μs, or by about 10 μs, or by about 15 μs, or other amounts as determined suitable. In step 276, the newly-adjusted parameters are again checked for device compliance, and steps 272, 274, 276 are repeated until the operating parameters are within compliance limits of the implantable system and in accordance with any other desired criteria, as mentioned above, and step 210 is then complete. Alternatively, if device compliance is verified in steps 224 and 254 as described above, step 210 may be omitted.

Referring now to FIG. 10, steps 212 and 214 and their associated sub-steps are depicted in further detail. The programming device 64 prompts the control system of the implanted baroreflex activation system to deliver a control signal to the electrode, which in turn activates the baroreflex system of the patient, wherein the control signal includes the operating parameters as determined by steps 206, 208, and 210 described above. In step 212, one or more patient hemodynamic measurements are obtained. Such measurements may comprise blood pressure heart rate, respiration, or any of the measurements described above with relation to step 202. If it is determined from the one or more patient hemodynamic measurements that the desired response is suitable, the programming procedure is complete. If it is determined from the one or more patient hemodynamic measurements that the desired response from the delivered therapy is less than desired in step 280, the programming device 64 is utilized to increase the magnitude of the pulse frequency in step 282, and the adjusted settings are programmed into the implanted device in step 284. If it is determined from the one or more patient hemodynamic measurements that the desired response from the delivered therapy is greater than desired in step 281, the programming device 64 is utilized to decrease the magnitude of the pulse frequency in step 283, and the adjusted settings are programmed into the implanted device in step 285.

A programming device 64 for use with the present invention generally includes a user interface and a display 65 which may be combined as a touchscreen interface or which may be separate such as a keypad and display. The user interface may be as depicted in FIG. 11, although other arrangements of the user interface are contemplated and within the spirit of the present invention. The programming device is configured to adjust any of the operating parameters described herein (such as pulse width, pulse amplitude, pulse frequency, etc.) as well provide an indication of the magnitude of the operating parameters; track the history of the operating parameters during the programming process; provide an indication of a patient response to a delivered therapy, such as by way of indicating an actual value for a patient physiological parameter or simply indicating favorable or unfavorable response; and provide indications of predicted battery life based on selected operating parameters. The programming device may prompt the user with step-by-step instructions to perform the programming steps as described herein, or the programming device may offer a manual operation mode, allowing a user to tailor the programming process as desired such as skipping any or all steps.

In one embodiment, tracking the history of the operating parameters during the programming process may comprise plotting the parameters as they are tested, with for example a first operating parameter along a first axis and a second operating parameter along a second axis. In another embodiment, the programming device may store values from past programming sessions.

Referring now to an alternate embodiment of programming method 200, the order of steps 206 and 208 are reversed such that step 208 is carried out prior to step 206. In such an embodiment, step 208 comprises setting a nominal value for pulse amplitude, such as 0.8 mA or 1.0 mA, and then adjusting the pulse width according to the procedures described above. Subsequently, in step 206 the pulse amplitude is adjusted according to the procedures described above.

In an alternate embodiment, steps 206 and 208 are carried out simultaneously, such that if the patient is not experiencing extraneous stimulation, both pulse amplitude and pulse width are increased at the same time before continuing to deliver therapy signals and check for patient response. In such an embodiment, the width and amplitude are increased from the initial value in a predetermined ratio until extraneous is encountered or compliance is exceeded. The amplitude and width are then simultaneously reduced in a predetermined ratio to eliminate extraneous, stay within compliance limits, and provide reasonable longevity as determined by the user. The ratio may be in the range of 0.1-1 mA for every 1-15 us. A preferred ratio includes increments of 0.4 mA for every 5 us. This approach has the advantage of determining programmed parameters with only a single control.

In an alternate embodiment, pulse width is set to an estimated value, the estimated value being greater than the initial value described above, and pulse amplitude is then increased in increments as described in conjunction with step 206, such that step 208 is effectively eliminated. Steps 210, 212 and 214 are then carried out as described above.

In an alternate embodiment, pulse amplitude is set to an estimated value, the estimated value being greater than initial value described above, and pulse width is then increased in increments as described in conjunction with step 208, such that step 206 is effectively eliminated. Steps 210, 212 and 214 are then carried out as described above.

In another embodiment, the programming method may take into account projected device longevity (e.g., useful service life of the battery of the implantable pulse generator) when setting the various operating parameters. For example, programming device 64 is configured to calculate the projected or predicted device longevity based on one or more parameters including but not limited to battery capacity, pulse amplitude, pulse width, and pulse frequency. In one embodiment, programming device 64 may provide an indication to a user of projected device longevity during the programming procedure, such that the user may choose whether or not to modify an operating parameter based on the indication of device longevity provided by programming device 64.

In another embodiment, programming device 64 may provide the option to select a mode of operation which can increase device longevity, such as selecting to deliver burst therapy or periodic therapy, wherein therapy is delivered for a given period of time and then not delivered for a period of time before being delivered again. Such an arrangement may maintain suitable therapeutic effect of the delivered therapy while extending device longevity.

In another embodiment, the user may input a desired device longevity into programming device 64. For example, the user may input a desired device longevity at the beginning of programming method 200, and then proceed with method 200 as described above and in FIGS. 6-10. In conjunction with one or more steps of programming method 200, an indication of device longevity is provided to the user and may thus influence the selected parameters. In another example, the user inputs a desired device longevity at the beginning of programming method 200 and programming device provides suggested values for one or more device operating parameters such as pulse amplitude, pulse width, pulse frequency, or others. The programming method may then begin by testing the suggested values based on the desired device longevity and then adjusted as needed.

It may be advisable when setting any of the operating parameters described herein to create a safety margin (factor) or buffer against extraneous stimulation, as the level at which a patient experiences extraneous stimulation may vary according to activity, posture, stress, environmental factors, physiological fluctuations or other factors. In one embodiment, the safety margin or buffer may be expressed as a percentage, such that if a patient experiences extraneous stimulation at a programmed value of X, the safety margin is defined as 90% of X (thereby creating a buffer of 10%). Alternate magnitudes for the buffer may be in the range of 5% to 25%.

Alternately, the safety margin or buffer may be expressed as a numerical value, such that if a patient experiences extraneous stimulation at a programmed value of Y, the safety margin is defined as Y minus Z, wherein Z is the buffer. In one embodiment, the buffer may be the same as the increments by which the various operating parameters are adjusted during programming as described above in conjunction with step 226 (for example, 1 mA), and step 256 (for example, 15 μs). In one embodiment, the buffer may be based on historical data obtained from prior programming procedures for the same patient and/or other patients, and such historical data may be relied upon to set or at least influence the buffer setting for the present patient. Optionally, the system may recommend parameter settings for pulse width and/or pulse amplitude using extraneous and/or historically-obtained buffer data which simultaneously maximize proximity to the buffer (and therefore maximize effectiveness of therapy) and maximize device longevity.

In one embodiment, programming device 64 may include an auto-programming feature, in which programming device performs a number of tests with implantable pulse generator 60 to determine suitable operating parameters. Programming device 64 may be operably coupled to a means for measuring at least one patient hemodynamic response and means for measuring a response indicative of extraneous stimulation. Programming device 64 delivers therapy signals using for example a preprogrammed series of therapy doses, and measures both the hemodynamic response of the patient as well as responses indicative of extraneous stimulation. In one embodiment, the response indicative of extraneous stimulation comprises the patient indicating a sensation or pain or lack thereof with each therapy pulse delivered. In one embodiment, the hemodynamic measurement is a sensor connected to the system, such as a blood pressure sensor. In one embodiment, the measurement is manually input from an external sensor measurement, such as an external blood pressure cuff. The measurements may be optionally displayed or plotted on the interface for the user as curves. A dose-response test may include one or a plurality of curves, each curve representing a therapy dose and subsequent patent blood pressure decrease. The system then determines, based on the hemodynamic response and extraneous measurement, a therapy target zone defined as the space between the dose-response and extraneous curves. The system displays one or several suggested parameters within the target zone to the user to program therapy for the patient. The user may then choose to accept, deny, or modify the suggested parameters.

In one embodiment, the present invention may comprise a kit which includes an implantable baroreflex activation system, a programming device, and a set of instructions recorded on a tangible medium for implanting, programming and/or operating the system. The contents of the kit may be provided in one or more hermetically sealed and sterilized packages. The implantable baroreflex activation system includes an implantable pulse generator within a hermetically sealed housing, a baroreflex activation device in the form of an electrode structure coupled to the pulse generator via a lead, and configured to be implantable proximate a baroreceptor of a patient. The programming device is as described elsewhere herein, and is configured to communicate with the implantable pulse generator for the purpose of programming and/or adjusting one or more operating parameters of the baroreflex activation system for the purpose of delivering a baroreflex activation therapy to a patient. The instructions may be recorded on a tangible medium, or indications may be provided linking a user to electrically accessible instructions.

Various modifications to the embodiments of the inventions may be apparent to one of skill in the art upon reading this disclosure. For example, persons of ordinary skill in the relevant art will recognize that the various features described for the different embodiments of the inventions can be suitably combined, un-combined, and re-combined with other features, alone, or in different combinations, within the spirit of the invention. Likewise, the various features described above should all be regarded as example embodiments, rather than limitations to the scope or spirit of the inventions. Therefore, the above is not contemplated to limit the scope of the present inventions.

For example, although the present invention is discussed mainly in reference to baroreflex activation systems, baroreflex devices, and implantable pulse generators in the context of such systems and devices; it is applicable to neurostimulators. It should be further appreciated by those skilled in the art that although the present invention is discussed and is of particular relevance to implantable pulse generators, it is also applicable to external pulse generators. Thus, the present invention and all embodiments described herein are applicable to pulse generators which are external (and not implantable) as well as those which are implantable. It should be further understood by those skilled in the art that the methods, devices, and systems according to the present invention are further applicable to modifying any one or more of the nervous system activity of the patient, autonomic nervous system activity of the patient, sympathetic and/or parasympathetic nervous system activity of the patient, or metabolic activity of the patient.

Persons of ordinary skill in the relevant arts will recognize that the inventions may comprise fewer features than illustrated in any individual embodiment described above. The embodiments described herein are not meant to be an exhaustive presentation of the ways in which the various features of the inventions may be combined. Accordingly, the embodiments are not mutually exclusive combinations of features; rather, the inventions may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the embodiments of the present inventions, it is expressly intended that the provisions of Section 112, sixth paragraph of 35 U.S.C. are not to be invoked unless the specific terms “means for” or “step for” are recited in a claim.

1. A method of programming a therapy for an implanted baroreflex stimulation system, the implanted baroreflex stimulation system configured to deliver the therapy to a patient, the therapy having at least two programmable operating parameters, the method comprising:
determining a first programmable operating parameter of the therapy, including:
delivering a first therapy pulse to the patient, the therapy pulse including initial settings for each of the at least two programmable operating parameters;
increasing the first programmable operating parameter in increments;
delivering subsequent therapy pulses at each increment;
determining, for at least some of the therapy pulses, whether the patient is experiencing extraneous stimulation associated with the therapy pulses; and
selecting a value for the first programmable operating parameter based at least in part on the last therapy pulse delivered which did not cause the patient to experience extraneous stimulation associated with the therapy pulse;
determining a second programmable operating parameter of the therapy, including:
delivering a further therapy pulse to the patient, the therapy pulse including the selected value for the first programmable operating parameter and the initial setting for the second programmable operating parameter;
increasing the second programmable operating parameter in increments;
delivering subsequent therapy pulses at each increment;
determining, for at least some of the therapy pulses, whether the patient is experiencing extraneous stimulation associated with the therapy pulses; and
selecting a value for the second programmable operating parameter based at least in part on the last therapy pulse delivered which did not cause the patient to experience extraneous stimulation associated with the therapy pulse; and
programming the implanted baroreflex stimulation system with the selected values for the first and second programmable operating parameters.

2. The method of claim 1, wherein the first programmable operating parameter comprises pulse amplitude, and wherein the second programmable operating parameter comprises pulse width.

3. The method of claim 1, wherein the first programmable operating parameter comprises pulse width, and wherein the second programmable operating parameter comprises pulse amplitude.

4. The method of claim 1, further comprising:
obtaining one or more patient hemodynamic measurements after selecting values for the first and second programmable operating parameters;
confirming, denying, or otherwise modifying one or more of the selected values for the first and second programmable operating parameters based on the one or more patient hemodynamic measurements.

5. The method of claim 1, wherein the initial settings for each of the at least two programmable operating parameters are pre-programmed into the implantable baroreflex activation system.

6. The method of claim 1, wherein the implanted baroreflex stimulation system includes a compliance limit for one or more of the first and second programmable operating parameters, the method further comprising:
comparing the selected values for at least one of the first and second programmable operating parameters against the compliance limit of the implanted baroreflex stimulation system; and
confirming, denying, or otherwise modifying one or more of the selected values for the first and second programmable operating parameters based on the compliance limit.

7. The method of claim 1, wherein the therapy includes a third programmable operating parameter comprising pulse frequency, the method further comprising:
adjusting the pulse frequency of the therapy in increments;
delivering subsequent therapy pulses at each increment;
obtaining one or more patient hemodynamic measurements after at least some of the therapy pulses;
selecting a value for the pulse frequency based at least in part on the one or more patient hemodynamic measurements.

8. The method of claim 1, wherein the therapy includes a third programmable operating parameter comprising pulse frequency, and wherein a magnitude of the pulse frequency has an effect on a longevity of a battery of the implanted baroreflex stimulation system, the method further comprising:
adjusting the pulse frequency of the therapy in increments;
determining, for each increment, an effect on the longevity of the battery of the implanted baroreflex stimulation system; and
selecting a value for the pulse frequency based at least in part on the effect on the longevity of the battery of the implanted baroreflex stimulation system.

9. The method of claim 1, wherein the first programmable operating parameter and the second programmable operating parameter are determined in conjunction, the method further comprising:
delivering a first therapy pulse to the patient, the therapy pulse including initial settings for each of the at least two programmable operating parameters;
increasing the first programmable operating parameter and the second programmable parameter in increments;
delivering subsequent therapy pulses at each increment;
determining, for at least some of the therapy pulses, whether the patient is experiencing extraneous stimulation associated with the therapy pulses; and
selecting a value for the first programmable operating parameter and a value for the second programmable operating parameter based at least in part on the last therapy pulse delivered which did not cause the patient to experience extraneous stimulation associated with the therapy pulse;
programming the implanted baroreflex stimulation system with the selected values for the first and second programmable operating parameters.

10. The method of claim 1, further comprising:
applying a predetermined safety factor to the selected value of at least one of the first and second programmable operating parameters so as to create a reduced value for the parameter; and
programming the implanted baroreflex stimulation system with the selected values for the first and second programmable operating parameters, including any reduced values.

11. The method of claim 1, further comprising:
causing the implanted baroreflex stimulation system to deliver a chronic therapy to the patient, the therapy including the selected values for the first and second programmable operating parameters.

12. A method of programming a therapy for an implanted baroreflex stimulation system, the system configured to deliver the therapy to a patient and including a subcutaneously-implanted pulse generator and a baroreflex activation device in the form of an electrode structure coupled to the pulse generator via a lead, the baroreflex activation device implanted such that the electrode structure is proximate a baroreceptor of a patient, the therapy having at least two programmable operating parameters, the method comprising:
causing a programming device to be manufactured and made available to a user, the programming device communicable with the implanted baroreflex stimulation system; and
providing instructions to the user, the instructions including:
determining a first programmable operating parameter of the therapy, including:
delivering a first therapy pulse to the patient, the therapy pulse including initial settings for each of the at least two programmable operating parameters;
increasing the first programmable operating parameter in increments;
delivering subsequent therapy pulses at each increment;
determining, for at least some of the therapy pulses, whether the patient is experiencing extraneous stimulation associated with the therapy pulses; and
selecting a value for the first programmable operating parameter based at least in part on the last therapy pulse delivered which did not cause the patient to experience extraneous stimulation associated with the therapy pulse;
determining a second programmable operating parameter of the therapy, including:
delivering a further therapy pulse to the patient, the therapy pulse including the selected value for the first programmable operating parameter and the initial setting for the second programmable operating parameter;
increasing the second programmable operating parameter in increments;
delivering subsequent therapy pulses at each increment;
determining, for at least some of the therapy pulses, whether the patient is experiencing extraneous stimulation associated with the therapy pulses; and
selecting a value for the second programmable operating parameter based at least in part on the last therapy pulse delivered which did not cause the patient to experience extraneous stimulation associated with the therapy pulse; and
programming the implanted baroreflex stimulation system with the selected values for the first and second programmable operating parameters.

13. The method of claim 12, the instructions further comprising:
causing the implanted baroreflex stimulation system to deliver a chronic therapy to the patient, the therapy including the selected values for the first and second programmable operating parameters.

14. The method of claim 12, wherein the first programmable operating parameter comprises pulse amplitude, and wherein the second programmable operating parameter comprises pulse width.

15. The method of claim 12, wherein the first programmable operating parameter comprises pulse width, and wherein the second programmable operating parameter comprises pulse amplitude.

16. The method of claim 12, wherein the therapy includes a third programmable operating parameter comprising pulse frequency, the instructions further including:
adjusting the pulse frequency of the therapy in increments;
delivering subsequent therapy pulses at each increment;
obtaining one or more patient hemodynamic measurements after at least some of the therapy pulses;
selecting a value for the pulse frequency based at least in part on the one or more patient hemodynamic measurements.

17. The method of claim 12, wherein the therapy includes a third programmable operating parameter comprising pulse frequency, and wherein a magnitude of the pulse frequency has an effect on a longevity of a battery of the implanted baroreflex stimulation system, the method further comprising:
adjusting the pulse frequency of the therapy in increments;
determining, for each increment, an effect on the longevity of the battery of the implanted baroreflex stimulation system; and
selecting a value for the pulse frequency based at least in part on the effect on the longevity of the battery of the implanted baroreflex stimulation system.

18. The method of claim 12, wherein the first programmable operating parameter and the second programmable operating parameter are determined in conjunction, the instructions further including:
delivering a first therapy pulse to the patient, the therapy pulse including initial settings for each of the at least two programmable operating parameters;
increasing the first programmable operating parameter and the second programmable parameter in increments;
delivering subsequent therapy pulses at each increment;
determining, for at least some of the therapy pulses, whether the patient is experiencing extraneous stimulation associated with the therapy pulses; and
selecting a value for the first programmable operating parameter and a value for the second programmable operating parameter based at least in part on the last therapy pulse delivered which did not cause the patient to experience extraneous stimulation associated with the therapy pulse;
programming the implanted baroreflex stimulation system with the selected values for the first and second programmable operating parameters.

19. The method of claim 12, the instructions further including:
applying a predetermined safety factor to the selected value of at least one of the first and second programmable operating parameters so as to create a reduced value for the parameter; and
programming the implanted baroreflex stimulation system with the selected values for the first and second programmable operating parameters, including any reduced values.

20. A programming device for programming a therapy for an implanted baroreflex stimulation system, the implanted baroreflex stimulation system configured to deliver the therapy to a patient, the therapy having at least two programmable operating parameters, the programming device communicably coupled to the implanted baroreflex stimulation system and configured to:
determine a first programmable operating parameter of the therapy, including:
deliver a first therapy pulse to the patient, the therapy pulse including initial settings for each of the at least two programmable operating parameters;
increase the first programmable operating parameter in increments;
deliver subsequent therapy pulses at each increment;
determine, for at least some of the therapy pulses, whether the patient is experiencing extraneous stimulation associated with the therapy pulses; and
select a value for the first programmable operating parameter based at least in part on the last therapy pulse delivered which did not cause the patient to experience extraneous stimulation associated with the therapy pulse;
determine a second programmable operating parameter of the therapy, including:
deliver a further therapy pulse to the patient, the therapy pulse including the selected value for the first programmable operating parameter and the initial setting for the second programmable operating parameter;
increase the second programmable operating parameter in increments;
deliver subsequent therapy pulses at each increment;
determine, for at least some of the therapy pulses, whether the patient is experiencing extraneous stimulation associated with the therapy pulses; and
select a value for the second programmable operating parameter based at least in part on the last therapy pulse delivered which did not cause the patient to experience extraneous stimulation associated with the therapy pulse; and
program the implanted baroreflex stimulation system with the selected values for the first and second programmable operating parameters.
